(19) United States
(12) Patent Application Publication
(10) Pub. No.: US 2024/0225625 A1
(43) Pub. Date: Jul. 11, 2024

(54) APPARATUS FOR USE WITH A SURGICAL INSTRUMENT

(71) Applicant: GYRUS MEDICAL LIMITED, St Mellons (GB)

(72) Inventor: David MORRIS, St Mellons (GB)

(73) Assignee: GYRUS MEDICAL LIMITED, St Mellons (GB)

(21) Appl. No.: 18/403,868

(22) Filed: Jan. 4, 2024

Related U.S. Application Data
(60) Provisional application No. 63/438,096, filed on Jan. 10, 2023.

(51) Int. Cl. A61B 17/00

(52) U.S. Cl. CPC A61B 17/00; A61B 2017/00398; A61B 2017/0046; A61B 2217/005

(57) ABSTRACT

An apparatus for use with a surgical instrument having a hollow shaft for transferring fluid and/or debris generated during use of the surgical instrument. The apparatus comprises: a drive unit comprising a driving part configured to generate a driving force for driving the surgical instrument, and a driven part driven by the driving part to transfer the driving force to the surgical instrument; and a channel arranged to receive the hollow shaft of the surgical instrument, the channel extending through the driven part of the drive unit.

### TECHNICAL FIELD

Embodiments of the present invention described herein relate to an apparatus and a corresponding surgical instrument. Embodiments of the present invention described herein also relate to a surgical device and a surgical system.

### BACKGROUND TO THE INVENTION AND PRIOR ART

Powered surgical devices, such as shavers, generate fluid and/or debris (e.g. tissue debris) during use. This fluid and/or debris can be removed from the surgical site along a suction lumen, using a suction force generated by a suction source. Powered surgical devices are commonly designed so that the flow path for the fluid and/or debris passes around the outside of the drive unit (e.g. a motor) of the surgical device.

### SUMMARY OF THE INVENTION

There are several disadvantages associated with providing a flow path around the outside of the drive unit. First, the flow path through the device is relatively convoluted, which leads to locations which can become clogged or blocked with debris during use of the device, and these locations may be difficult to access to clean after use. If additional heat sources (e.g. radio frequency (RF) devices) are used, the temperature of the fluid (e.g. saline) is higher, and it is possible for the drive unit to become hot during use of the heat source. This can compromise the life and performance of the drive unit. Warm fluid passing around the drive unit can also cause the temperature of the outer surface of the device to increase, which is not desirable for the user. Also, providing the flow path around the outside of the drive unit leads to a relatively large device for the user to hold, and such a device is often not ergonomic.

The present invention addresses these issues by providing a surgical device which is configured so that the flow path extends through the middle of the drive unit rather than around the outside of the drive unit. In particular, the flow path extends through a driven part of the drive unit (e.g. the rotary shaft in a motor). This arrangement provides several advantages. First, the flow path for the fluid and/or debris through the device is straight, reducing the chance of debris collecting at points within the flow path. There is also more space within the device for electrical connections (e.g. for transferring power or sensing), and the device is more compact. Any warm fluid passing through the hollow shaft of the surgical instrument flows through the drive unit rather than around the outside of the drive unit, thereby preventing heat from being transferred to the user via the outer surface of the device.

In view of the above, from one aspect the present invention provides an apparatus for use with a surgical instrument having a hollow shaft for transferring fluid and/or debris generated during use of the surgical instrument. The apparatus comprises: a drive unit comprising a driving part configured to generate a driving force for driving the surgical instrument, and a driven part driven by the driving part to transfer the driving force to the surgical instrument; and a channel arranged to receive the hollow shaft of the surgical instrument, the channel extending through the driven part of the drive unit.

The drive unit may comprise a motor and the driven part may comprise a hollow drive shaft. The channel may extend through the drive shaft.

The drive unit may comprise a first coupling portion disposed at a distal end of the drive unit, the first coupling portion being connectable to the surgical instrument so as to transmit the driving force from the drive unit to the surgical instrument.

The first coupling portion may comprise a recess arranged to receive a coupler of the surgical instrument.

The apparatus may comprise a secondary channel connecting the channel to a proximal end of the apparatus.

The apparatus may comprise a second coupling portion disposed at the proximal end of the apparatus, the second coupling portion being connectable to a suction tube.

The apparatus may comprise a flow valve disposed in the secondary channel.

The apparatus may have a recess at its distal end, the recess being arranged to receive a hub of the surgical instrument.

The apparatus may have a longitudinal axis, and the channel may extend along the longitudinal axis.

In another aspect, the invention provides a surgical instrument comprising: a hollow shaft for transferring fluid and/or debris generated during use of the surgical instrument; and a hub comprising: a body; a first coupler fixed to the hollow shaft, wherein the first coupler is connectable to a drive unit so as to receive driving force from the drive unit and transfer the driving force to the hollow shaft; and a first rotary seal between the first coupler and the body.

The first coupler may be insertable into a recess of the drive unit.

The surgical instrument may comprise a second coupler disposed at a proximal end of the hollow shaft.

The surgical instrument may comprise a second rotary seal disposed around the second coupler.

In another aspect, the invention provides a surgical device including an apparatus and a surgical device as set out above.

In another aspect, the invention provides a surgical system comprising a surgical device as set out above, and a suction source connectable to the surgical device.

### BRIEF DESCRIPTION OF THE DRAWINGS

Embodiments of the invention will now be further described by way of example only and with reference to the accompanying drawings, wherein like reference numerals refer to like parts, and wherein:

FIG. 1 is a schematic diagram of a surgical system including a surgical device according to an embodiment;

FIG. 2 is a side view of a surgical device according to an embodiment;

FIG. 3A is a side view of an apparatus according to an embodiment, and

FIG. 3B is a cross-sectional side view of the apparatus;

FIG. 4A is a side view of a surgical instrument according to an embodiment, and FIG. 4B is a cross-sectional side view of the surgical instrument;

FIG. 5 is a cross-sectional side view of a hub of the surgical instrument; and

FIGS. 6A and 6B are cross-sectional side views of the surgical instrument and the apparatus connected together.

### DESCRIPTION OF THE EMBODIMENTS

An apparatus is described herein in the context of a surgical device for performing procedures such as ablation, sealing, resection and coagulation of tissues. The surgical device is described herein by way of example as an electrosurgical device.

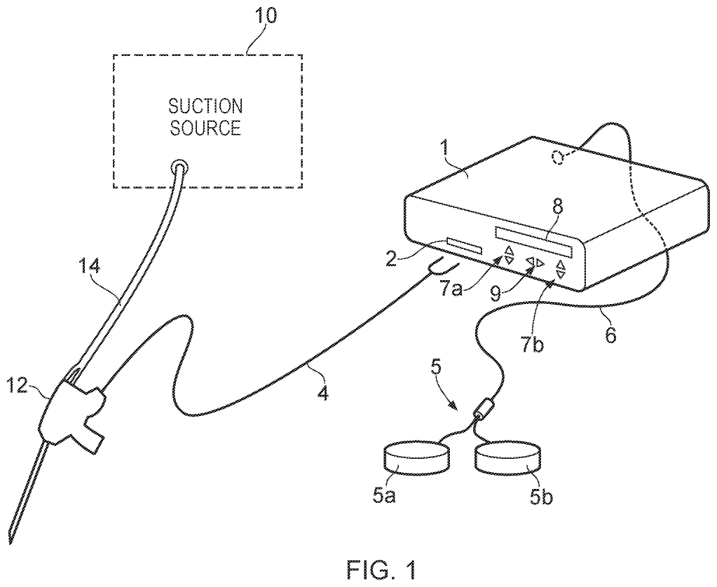

Referring to the drawings, FIG. 1 shows an electrosurgical generator 1 having an output socket 2 providing a radio frequency (RF) output, via a connection cord 4, for an electrosurgical device 12. The device 12 has irrigation and suction tubes 14 which are connected to an irrigation fluid and suction source 10. Activation of the generator 1 may be performed from the device 12 via a handswitch (not shown) on the device 12, or by means of a footswitch unit 5 connected separately to the rear of the generator 1 by a footswitch connection cord 6. In the embodiment shown in FIG. 1, the footswitch unit 5 has two footswitches 5a and 5b for selecting a coagulation mode or a cutting or vaporisation (ablation) mode of the generator 1 respectively. The generator front panel has push buttons 7a and 7b for respectively setting ablation (cutting) or coagulation power levels, which are indicated in a display 8. Push buttons 9 are provided as an alternative means for selection between the ablation (cutting) and coagulation modes.

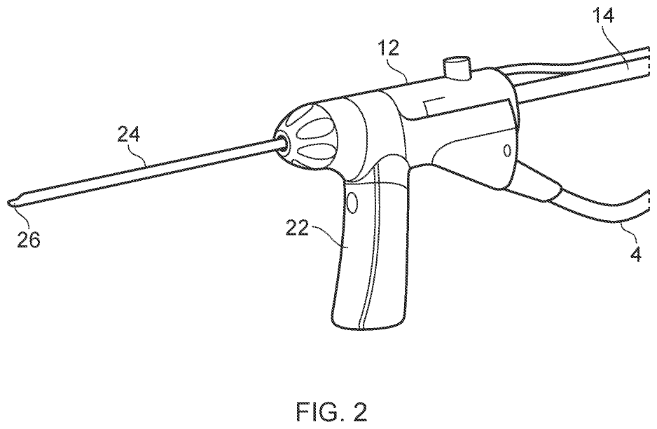

FIG. 2 shows a surgical device 12 according to an embodiment of the present invention. In the present embodiment, the surgical device is an electrosurgical device. The device 12 includes a proximal handle portion 22 which includes a drive unit (not shown). The device 12 also includes a surgical instrument including a fixed hollow outer shaft 24 extending in a distal direction away from the proximal handle portion 22, and a distal end effector assembly 26 at the distal end of the outer shaft. A hollow inner shaft (not shown) is disposed within the hollow shaft 24 and transfers fluid and/or debris generated during use of the surgical instrument. The hollow inner shaft can rotate within the hollow outer shaft during use of a shaving function.

A power connection cord 4 connects the device to the RF generator 1, whereas tubes 14 connect the device to the irrigation and suction source 10. The device may further be provided with activation buttons (not shown), to allow the surgeon operator to activate either the mechanical cutting function of the end effector, or the electrosurgical functions of the end effector, which in this embodiment typically comprise coagulation or ablation.

Examples of the apparatus and surgical instrument shown in FIGS. 1 and 2 will now be described.

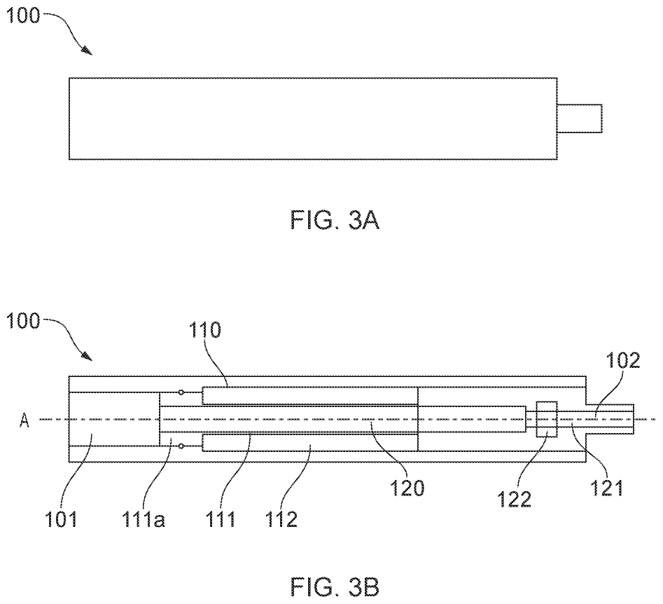

An example of the apparatus is shown in FIGS. 3A and 3B. The apparatus 100 includes a drive unit configured to generate a driving force for driving the surgical instrument. In the present example, the drive unit is a motor 110. The motor 110 includes a hollow drive shaft 111, which is the driven part of the motor 110, and a stator 112, which is the driving part of the motor 110. The stator 112 surrounds the drive shaft 111. In other examples, the drive unit may be, for example, an oscillator.

The apparatus 100 is substantially cylindrical in shape, and includes a main channel 120 which extends through the apparatus 100 along a longitudinal axis A of the apparatus 100. In particular, the channel 120 extends through the drive shaft 111 of the motor 110. The channel 120 is arranged to receive a hollow shaft of the surgical instrument. This allows fluid and/or debris to be transferred through the middle of the motor 110 when the hollow shaft of the surgical instrument is inserted in the channel 120 in use. This arrangement provides several advantages. First, the flow path for the fluid and/or debris through the apparatus 100 is straight, reducing the chance of debris collecting at points within the flow path. There is also more space within the apparatus 100 for electrical connections (e.g. for transferring power or sensing), and the apparatus is more compact. Any warm fluid passing through the hollow shaft of the surgical instrument flows through the motor 110 rather than around the outside of the motor 110, thereby preventing heat from being transferred to the user via the outer surface of the apparatus 100.

The drive shaft 111 of the motor 110 includes a coupling portion 111a disposed at the distal end of the drive shaft 111. The coupling portion 111a is connectable to the surgical instrument, so as to transmit the driving force from the motor 110 to the surgical instrument. In the present example, the coupling portion 111a includes a recess arranged to receive a coupler of the surgical instrument.

The apparatus 100 has a recess 101 at its distal end. The recess 101 is arranged to receive a hub of the surgical instrument. The proximal end of the apparatus 100 is defined by a coupling portion 102. A secondary channel 121 extends between the proximal end of the main channel 120 and the proximal end of the apparatus 100. The coupling portion 102 is connectable to a suction tube which connects the apparatus 100 to a suction source, such as the suction source 10 shown in FIG. 1.

In the present example, a flow valve 122 is disposed in the secondary channel 121, after the motor 110 in the flow path. The flow valve 122 is configured to control the rate of flow of fluid through the apparatus 100. Since the flow valve 122 is disposed separately from the motor 110, the valve can be cleaned during use if required. In other examples, the flow valve may be omitted. In such examples, the rate of flow of fluid through the apparatus can be controlled by the external suction source.

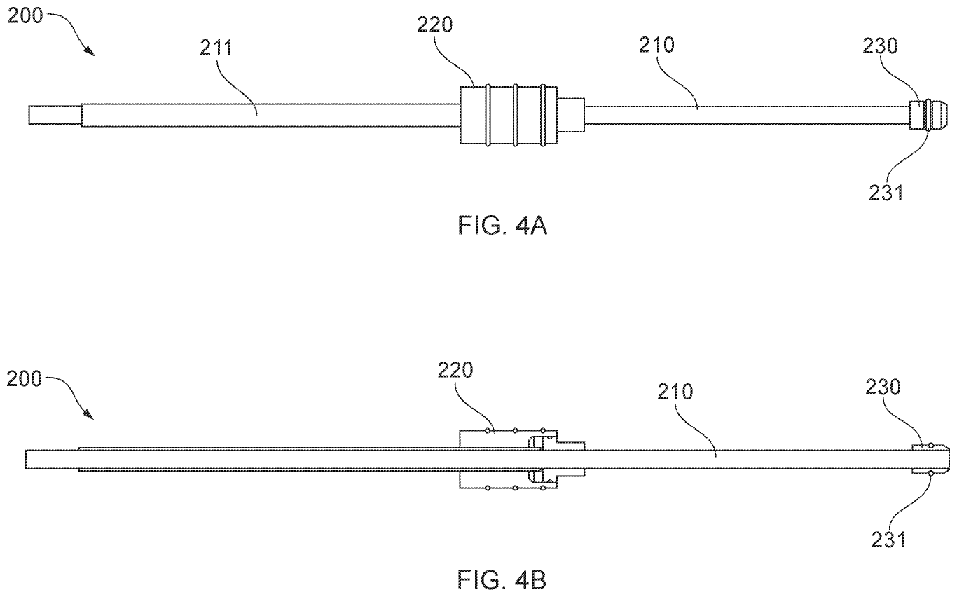

An example of the surgical instrument is shown in FIGS. 4A and 4B. The surgical instrument 200 includes a hollow inner shaft 210 for transferring fluid and/or debris generated during use of the surgical instrument. The hollow inner shaft 210 is disposed within a hollow outer shaft 211.

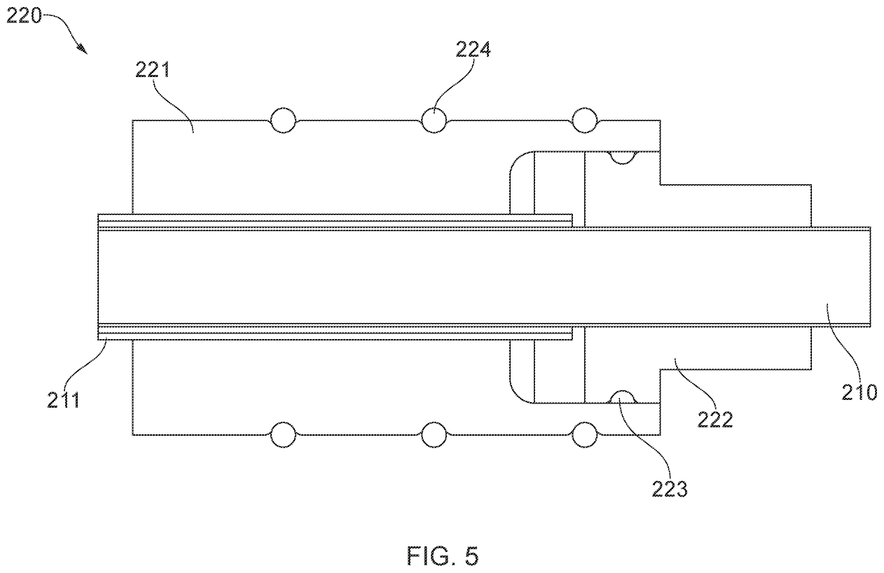

The surgical instrument 200 also includes a hub 220, which is shown in detail in FIG. 5. As shown in this figure, the hub 220 includes a body 221 and a coupler 222 which is received within a recess at the proximal end of the body 221. The inner shaft 210 passes through the body 221 and the coupler 222, while the outer shaft 211 passes through the body 221 and terminates before the coupler 222. The inner shaft 210 is fixed to the coupler 222, e.g. by adhesive, press fitting or welding, so that the coupler 222 and the hollow shaft 210 rotate together in use. The outer shaft 211 is fixed to the body 221 in a similar manner

A rotary seal 223 is provided between the coupler 222 and the body 221. The rotary seal 223 prevents any ingress or egress of fluid between the body 221 and the coupler 222 while allowing the coupler 222 to rotate relative to the body 221. In addition, one or more rotary seals 224 (in the present case three rotary seals) are disposed around the outside of the body 221. These rotary seals 224 prevent any ingress or egress of fluid from/to the outside environment.

Referring again to FIGS. 4A and 4B, the surgical instrument 200 further includes a second coupler 230 disposed at the proximal end of the hollow inner shaft 210. A rotary seal 231 is disposed around the second coupler 230.

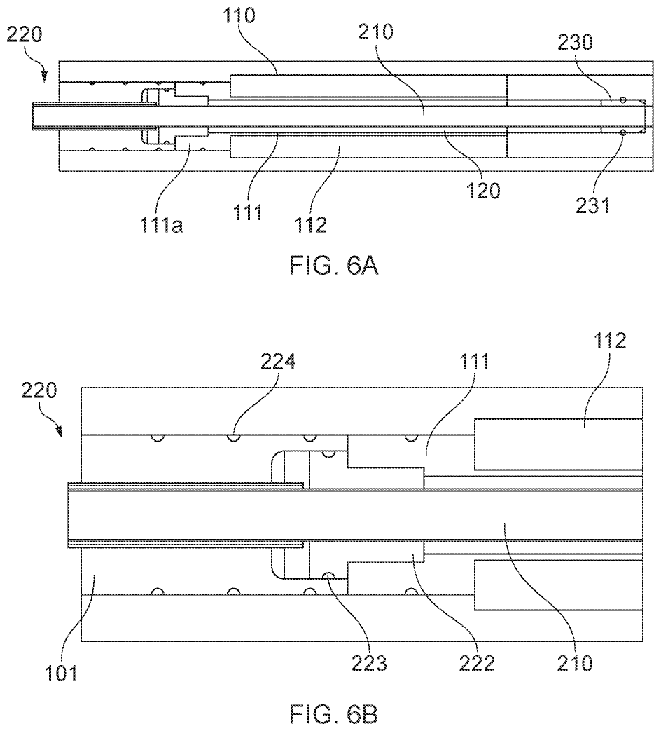

FIG. 6A shows the apparatus 100 and the surgical instrument 200 connected together. FIG. 6B shows a detailed view of the connection between the apparatus 100 and the surgical instrument 200. As shown in these figures, the hollow inner shaft 210 of the surgical instrument 200 is inserted in the channel 120 of the apparatus 100. The second coupler 230 of the surgical instrument abuts the proximal end of the main channel 120. The rotary seal 231 of the second coupler 230 prevents any ingress or egress of fluid between the second coupler 230 and the inner surface of the channel 120 while allowing the second coupler 230 to rotate within the channel 120.

The hub 220 is received in the recess 101 of the apparatus, and the first coupler 222 is received in the recess of the first coupling portion 111a of the drive shaft 111. A mechanical connection is formed between the first coupler 222 and the drive shaft 111, so that the driving force generated by the motor 110 can be transmitted to the hollow shaft 210 via the first coupler 222.

Various modifications whether by way of addition, deletion, or substitution of features may be made to above described embodiment to provide further embodiments, any and all of which are intended to be encompassed by the appended claims.

1. An apparatus for use with a surgical instrument having a hollow shaft for transferring fluid and/or debris generated during use of the surgical instrument, the apparatus comprising:
a drive unit comprising a driving part configured to generate a driving force for driving the surgical instrument, and a driven part driven by the driving part to transfer the driving force to the surgical instrument; and
a channel arranged to receive the hollow shaft of the surgical instrument, the channel extending through the driven part of the drive unit.

2. An apparatus according to claim 1, wherein the drive unit comprises a motor and the driven part comprises a hollow drive shaft, wherein the channel extends through the drive shaft.

3. An apparatus according to any claim 1, wherein the drive unit comprises a first coupling portion disposed at a distal end of the drive unit, the first coupling portion being connectable to the surgical instrument so as to transmit the driving force from the drive unit to the surgical instrument.

4. An apparatus according to claim 3, wherein the first coupling portion comprises a recess arranged to receive a coupler of the surgical instrument.

5. An apparatus according to claim 1, further comprising a secondary channel connecting the channel to a proximal end of the apparatus.

6. An apparatus according to claim 5, further comprising a second coupling portion disposed at the proximal end of the apparatus, the second coupling portion being connectable to a suction tube.

7. An apparatus according to claim 5, further comprising a flow valve disposed in the secondary channel.

8. An apparatus according to claim 1, wherein the apparatus has a recess at its distal end, the recess being arranged to receive a hub of the surgical instrument.

9. An apparatus according to claim 1, wherein the apparatus has a longitudinal axis, and the channel extends along the longitudinal axis.

10. A surgical instrument comprising:
a hollow shaft for transferring fluid and/or debris generated during use of the surgical instrument; and
a hub comprising:
a body;
a first coupler fixed to the hollow shaft, wherein the first coupler is connectable to a drive unit so as to receive driving force from the drive unit and transfer the driving force to the hollow shaft; and
a first rotary seal between the first coupler and the body.

11. A surgical instrument according to claim 10, wherein the first coupler is insertable into a recess of the drive unit.

12. A surgical instrument according to claim 10, further comprising a second coupler disposed at a proximal end of the hollow shaft.

13. A surgical instrument according to claim 12, further comprising a second rotary seal disposed around the second coupler.

14. A surgical device comprising:
i) an apparatus for use with a surgical instrument having a hollow shaft for transferring fluid and/or debris generated during use of the surgical instrument, the apparatus comprising: a drive unit comprising a driving part configured to generate a driving force for driving the surgical instrument, and a driven part driven by the driving part to transfer the driving force to the surgical instrument; and a channel arranged to receive the hollow shaft of the surgical instrument, the channel extending through the driven part of the drive unit;
the surgical device further comprising:
ii) the surgical instrument, comprising a hollow shaft for transferring fluid and/or debris generated during use of the surgical instrument; and a hub comprising: a body; a first coupler fixed to the hollow shaft, wherein the first coupler is connectable to a drive unit so as to receive driving force from the drive unit and transfer the driving force to the hollow shaft; and a first rotary seal between the first coupler and the body

15. A surgical system, comprising:
a surgical device according to claim 14; and
a suction source connectable to the surgical device source connectable to the surgical device.
